(19) United States
(12) Patent Application Publication
(10) Pub. No.: US 2023/0061908 A1
(43) Pub. Date: Mar. 2, 2023

(54) VSV (VITAMIN C-SPIRULINA-VITAMIN D) COMPOSITION, KIT AND METHOD

(71) Applicant: Joseph MILSTEIN, Lakewood, NJ (US)

(72) Inventor: Joseph MILSTEIN, Lakewood, NJ (US)

(21) Appl. No.: 17/978,418

(22) Filed: Nov. 1, 2022

Related U.S. Application Data
(63) Continuation of application No. PCT/IB2021/053732, filed on May 4, 2021.
(60) Provisional application No. 63/019,612, filed on May 4, 2020.

(51) Int. Cl. A61K 35/748; A61K 31/375; A61K 31/593; A61P 31/14

(52) U.S. Cl. CPC A61K 35/748; A61K 31/375; A61K 31/593; A61P 31/14

(57) ABSTRACT

A formulation comprises a combination of spirulina, vitamin C and vitamin D. In some versions (i) the spirulina comprises between 0.25X and 4X, wherein X is 452 milligrams, (ii) the vitamin C comprises between 0.25Y and 4Y, wherein Y is 298 milligrams, and (iii) the vitamin D comprises between 0.25Z and 4Z, wherein Z is 298 International Units (IU). A method of treating a Covid-19 coronavirus infection in a person, by administering to the person a daily dosage of a medicament that comprises spirulina, vitamin C and vitamin D, and a method of treating a pathogenic inflammation in a person by administering to the person a daily dosage of a medicament that comprises spirulina, vitamin C and vitamin D, are described. A method of treating asthma in a person by administering to the person a daily dosage of a medicament that comprises spirulina, vitamin C; and vitamin D is described.

### FIELD OF THE INVENTION

Certain embodiments are compositions, kits and methods directed to treat pathogenic inflammations or COVID-19 coronavirus infections or other viral infections in which there are multiple stages of the infection including a viral replication stage and an inflammatory stage.

### BACKGROUND

Coronavirus disease 2019 (COVID-19) is an infectious disease caused by severe acute respiratory syndrome coronavirus 2 (SARS-CoV-2). It was first identified in December 2019 in Wuhan, the capital of China's Hubei province, and has since spread globally, resulting in an ongoing pandemic. As of May 3, 2021, more than 152 million cases have been reported across 187 countries and territories, resulting in more than 3.2 million deaths.

Common symptoms include fever, cough, fatigue, shortness of breath, and loss of smell and taste. While the majority of cases result in mild symptoms, some progress to viral pneumonia, multi-organ failure, and cytokine storm. The time from exposure to onset of symptoms is typically around five days but may range from two to fourteen days.

Spirulina is a type of cyanobacteria, which grows in both fresh and salt water and is often known as blue-green algae. This microalga is very rich in nutrients, such as proteins, vitamins and minerals and is considered one of the most nutritious foods known.

### SUMMARY OF THE EMBODIMENTS

Applicant believes that Covid-19 has multiple stages. In the “incubation” stage, which may be 4-6 days, there may be no symptoms. In what may be called a second stage of the Covid-19 condition, the coronavirus multiplies in the patient's body and the body's immune system fights against the virus. During this second stage the patient experiences clinical symptoms. In the third stage, which may also be referred to herein as “post-viral”, the virus, for example the coronavirus, more specifically for example the Covid19 coronavirus, is weakening, inert or no longer viable but symptoms occur caused by the inflammation. The symptoms associated with a cytokine storm (a physiological reaction in which the patient's innate immune system causes an uncontrolled and excessive release of pro-inflammatory signaling molecules called cytokines) may consist of shortness of breath, coughing, congestion in the lungs, fever, chills, cardiac disfunction, muscle and joint aches.

As a result of the fact that in the first two stages there is a live virus whereas in the third stage the virus is weakened or inert, it is suggested that the medical condition that characterizes the first two stages is different from the medical condition that characterizes the third stage. Accordingly, applicant believes that a medicament to treat Covid-19 coronavirus and other viral infections having multiple stages that include a viral stage and an inflammatory stage should be configured to treat multiple stages of the condition so as to be most effective. In the case of the Covid-19, this would include both the first two active viral stages in which there is a virus and the third stage which is characterized primarily as an inflammatory condition with features of autoimmunity.

For a variety of reasons, it is not always practical to determine the exact stage that the Covid-19 patient's medical condition is in. Even if it were possible the patient has not always sought medical care at an early enough stage to know. Consequently, the patient and/or doctor/caregiver does not always immediately identify or know what stage the patient is in. Furthermore, the stages (for example the 2^{nd} and 3^{rd} stages) may overlap. These factors make it even more important for the treatment regime to be able to address multiple stages of the patients Covid-19 medical condition, and ideally to do so simultaneously to the extent possible without adverse effects. The same is true for other medical conditions, such as other viral infections, in which there is a viral stage and an inflammatory stage of the patient.

In certain embodiments, the combination of ingredients is able to treat damage and/or symptoms caused by each of the stages of Covid-19. In certain embodiments, the medicament has anti-viral properties, anti-inflammatory properties and immune modulation properties.

Applicant has found a multifunctional synergistic effect from the combination of spirulina, vitamin C and vitamin D. This effect is exploited herein for medical conditions in which there is a need for anti-viral and anti-inflammatory effects. This is particularly true for medical conditions in which there is a need for anti-viral, anti-inflammatory and immune modulation properties.

Applicant has found that in the combination of ingredients it is useful to use spirulina. Spirulina has powerful antioxidative and anti-inflammatory activities, which have many beneficial medicinal influences. It has antiviral activity against several viruses including herpes simplex virus type 2 (HSV-2), human cytomegalovirus (HCMV), and HSV-1. The anti-inflammatory activity is also associated with favorable effect on cholesterol, triglyceride and glucose metabolism.

Vitamin C is an essential vitamin. However, humans cannot produce Vitamin C and must rely on external sources, fruits and vegetables being the principal source. Vitamin C has numerous health benefits and is required for proper development and function of many functions of the body. It acts as a powerful antioxidant and plays an important role in maintaining proper immune function. This protects the body against infections of various kinds.

The function of Vitamin C can be beneficially modified by interaction with certain bioactive herbal and plant components, which by themselves have anti-inflammatory activity.

Vitamin D is produced in the skin in response to exposure to sunlight and can be obtained from certain foods. It carries many beneficial health effects including protection against heart disease. It is also associated with a positive effect on the immune system. Clinical research has demonstrated reduction in the incidence of seasonal influenza A, suggesting at least indirect antiviral activity.

Certain embodiments described herein successfully provide a composition directed to treat COVID-19 patients. The composition includes a combination, in some embodiments of specific concentrations, of vitamin C, spirulina and vitamin D which create a multifunctional synergistic effect of the three ingredients in treating COVID-19 patients.

In some embodiments, this combination of ingredients fights the disease in both the viral stage or stages and the inflammatory stages of the disease.

Moreover, there is scientific evidence that Covid-19 interferes with the blood clotting mechanism, thereby increasing the risk for thrombotic events. Applicant believes that the combination of the three ingredients, including spirulina, Vitamin C and vitamin D, is beneficial for the prevention of blood clotting, a serious condition associated with Covid-19, without generating a meaningful risk of hemorrhage.

Furthermore, and of great concern, is that the Covid-19 coronavirus causes damage to the central nervous system, which is reflected in a variety of neurological symptoms and dysfunction. Both the Covid-19 virus and structural damage in brain tissue has been documented. There is evidence that the Covid-19 virus penetrates the blood brain barrier (BBB). The BBB is a composition of microvascular endothelial cells, astrocytes, pericytes, and microglia, each having specific functions, but all of which are aimed at protecting the brain from invasion by injurious agents. Viruses have the capacity of entering the brain by damaging the tight junctions between cells of the BBB (paracellular invasion) and by being carried by infected phagocytes. Phagocytes, are immune cells, whose essential function is to destroy invading injurious agents in order to protect the organism from damage. They normally cross the BBB in order to fulfill their important protective function. However, if they are themselves infected by the virus, they may act as a transport vehicle making it possible for the virus to reach the brain,

Inflammation of the endothelial vascular wall, which is caused or increased by the covid-19 virus also increases the permeability of the BBB and facilitates viral entry. Anti-inflammatory substances in spirulina, Vitamin C and Vitamin D preserve the integrity of the cellular defense structures in the endothelial wall, which is thereby able to maintain its barrier function.

In the event that the virus has succeeded in passing through the BBB with the help of infected phagocytes, ordinary antiviral drugs are well known to be unable to penetrate the brain from the bloodstream since they are substrates of active efflux transporters. For example, it is known that Remdesivir (which is used to treat Covid-19) has wide distribution to most tissues, including the kidneys, liver, prostate gland, and salivary gland, but does not cross the BBB (blood-brain barrier). Likewise, Ivermectin (which is used to treat Covid-19) is not thought to readily cross the blood-brain barrier in humans either, since it is excluded by a P-glycoprotein drug pump (mdr-1). In contrast, in certain embodiments, the combination of spirulina, vitamin C and Vitamin D complex, is able to cross the BBB and is able to exert its antiviral and anti-inflammatory activity, thereby inhibiting the damaging effect of the virus.

One aspect is a formulation comprising a combination of spirulina, vitamin C and vitamin D.

In some embodiments, (i) the spirulina comprises between 0.25X and 4X, wherein X is 452 milligrams, (ii) the vitamin C comprises between 0.25Y and 4Y, wherein Y is 298 milligrams, and (iii) the spirulina comprises between 0.25Z and 4Z, wherein Z is 298 International Units (IU). In some embodiments, (i) the spirulina comprises between 0.5X and 2X, (ii) the vitamin C comprises between 0.5Y and 2Y, and (iii) the spirulina comprises between 0.5Z and 2Z. In some embodiments, a ratio of an amount of milligrams of vitamin C in the formulation to an amount of milligrams of spirulina in the formulation is between 1:2 and 5:6. In some embodiments, a ratio of the amount of IU of vitamin D in the formulation to an amount of milligrams of spirulina in the formulation is between 1:2 and 5:6.

In some embodiments, a ratio of an amount of milligrams of vitamin C in the formulation to an amount of milligrams of spirulina in the formulation is between 1:3 and 1:1, and a ratio of an amount of IU of vitamin D in the formulation to an amount of milligrams of spirulina in the formulation is between 1:3 and 1:1. In some embodiments, the ratio of the amount of milligrams of vitamin C in the formulation to the amount of milligrams of spirulina in the formulation is between 1:2 and 5:6 and wherein the ratio of the amount of IU of vitamin D in the formulation to the amount of milligrams of spirulina in the formulation is between 1:2 and 5:6. In some embodiments, the ratio of the amount of milligrams of vitamin C in the medicament to the amount of IU of vitamin D in the medicament to an amount of milligrams of spirulina in the medicament is between one-half and three-halves.

Another aspect is a kit or pack for treating a Covid-19 coronavirus, comprising one or a combination of tablets, caplets, capsules, pellets and/or lozenges that in combination include at least: 452 milligrams of spirulina; 298 milligrams of vitamin C; and 298 IU of vitamin D.

In some embodiments, the one or a combination of tablets, caplets, capsules, pellets and/or lozenges includes at least: 1356 milligrams of spirulina; 894 milligrams of vitamin C; and 894 IU of vitamin D.

In some embodiments, the one or a combination of tablets, caplets, capsules, pellets and/or lozenges includes: 1300 to 3200 milligrams of spirulina; 894 to 2100 milligrams of vitamin C; and 894 to 2100 IU of vitamin D.

Still another aspect is a method of treating a pathogenic inflammation in a person, comprising administering to the person for at least three days a daily dosage of a medicament that comprises: spirulina; vitamin C; and vitamin D.

In some embodiments, (i) the spirulina comprises between 0.25X and 4X, wherein X is 452 milligrams, (ii) the vitamin C comprises between 0.25Y and 4Y, wherein Y is 298 milligrams, and (iii) the vitamin D comprises between 0.25Z and 4Z, wherein Z is 298 International Units (IU).

In some embodiments, (i) the spirulina comprises between 0.5X and 2X, (ii) the vitamin C comprises between 0.5Y and 2Y, and (iii) the vitamin D comprises between 0.5Z and 2Z.

In some embodiments, a ratio of an amount of milligrams of vitamin C in the medicament to an amount of milligrams of spirulina in the medicament is between 1:2 and 5:6.

In some embodiments, a ratio of the amount of IU of vitamin D in the medicament to an amount of milligrams of spirulina in the medicament is between 1:2 and 5:6.

Another aspect is a method of treating a Covid-19 coronavirus infection in a person, comprising administering to the person for at least three days a daily dosage of a medicament that comprises: spirulina; vitamin C; and vitamin D.

In some embodiments, (i) the spirulina comprises between 0.25X and 4X, wherein X is 452 milligrams, (ii) the vitamin C comprises between 0.25Y and 4Y, wherein Y is 298 milligrams, and (iii) the vitamin D comprises between 0.25Z and 4Z, wherein Z is 298 International Units (IU).

In some embodiments, (i) the spirulina comprises between 0.5X and 2X, (ii) the vitamin C comprises between 0.5Y and 2Y, and (iii) the vitamin D comprises between 0.5Z and 2Z.

In some embodiments, a ratio of an amount of milligrams of vitamin C in the medicament to an amount of milligrams of spirulina in the medicament is between 1:2 and 5:6.

In some embodiments, a ratio of the amount of IU of vitamin D in the medicament to an amount of milligrams of spirulina in the medicament is between 1:2 and 5:6.

Yet still another aspect is a method of treating asthma in a person, comprising administering to the person for at least three days a daily dosage of a medicament that comprises: spirulina, wherein the spirulina is Arthrospira platensis; vitamin C; and vitamin D.

In some embodiments, (i) the spirulina comprises between 0.25X and 4X, wherein X is 452 milligrams, (ii) the vitamin C comprises between 0.25Y and 4Y, wherein Y is 298 milligrams, and (iii) the vitamin D comprises between 0.25Z and 4Z, wherein Z is 298 International Units (IU).

In some embodiments, (i) the spirulina comprises between 0.5X and 2X, (ii) the vitamin C comprises between 0.5Y and 2Y, and (iii) the vitamin D comprises between 0.5Z and 2Z.

In some embodiments, a ratio of an amount of milligrams of vitamin C in the medicament to an amount of milligrams of spirulina in the medicament is between 1:2 and 5:6.

In some embodiments, a ratio of the amount of IU of vitamin D in the medicament to an amount of milligrams of spirulina in the medicament is between 1:2 and 5:6.

These and other features, aspects and advantages of the invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is a flow chart of a method in accordance with one embodiment; and

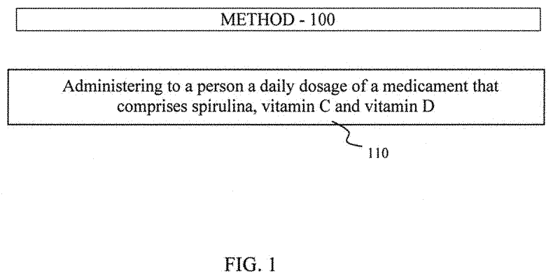

FIG. 2 is schematic view of a kit or pack, in accordance with one embodiment.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims. Accordingly, the invention is not necessarily limited in its application to the details of construction and the arrangement of the components and/or method set forth in the following description and/or illustrated in the drawings and/or Examples. The invention is capable of other embodiments or of being practiced or carried out in various ways.

Applicant has discovered a combination of ingredients that in certain embodiments are effective in both the first two viral stages and the third post-viral stage and possibly what is called “long-covid”. In certain embodiments, the combination of ingredients effectuates an anti-viral effect, an anti-inflammatory effect and an immune modulation effect.

In some embodiments, there is a composition configured to treat COVID-19 patients. In some embodiments a composition has three ingredients, for example vitamin C, spirulina and vitamin D. In certain embodiments, the compositions also includes a combination of specific concentrations of each ingredient, for example of vitamin C, spirulina, and vitamin D. The concentration of each component may vary in different embodiments and may be of different quantities. Furthermore, the composition may further include additions of, for example, stabilizers, preservatives, antioxidants, and the like.

In the case of Covid-19, the VSV composition can be administered to a COVID 19 patient, for example, orally or parenterally in the form of, for example, a pill (including but not limited to a tablet, capsule, caplet, lozenge, pellet), a liquid, etc. in measured doses.

The principles and operation of a VSV (Vitamin C-Spirulina-Vitamin D) composition, kit and method according to the invention may be better understood with reference to the drawings and the accompanying description.

One embodiment is a formulation, comprising spirulina, vitamin C and vitamin D, in one version for example where the spirulina is Arthrospira platensis. In one version of this, the vitamin C is a low dose of vitamin C, wherein the low dose of vitamin C is a daily dosage less than about 400 milligrams. The vitamin D may be vitamin D3.

One embodiment is a formulation comprising a combination of spirulina, vitamin C and vitamin D in which

(i) the spirulina comprises between 0.25X and 4X, wherein X is 452 milligrams,

(ii) the vitamin C comprises between 0.25Y and 4Y, wherein Y is 298 milligrams, and

(iii) the spirulina comprises between 0.25Z and 4Z, wherein Z is 298 International Units (IU).

In this embodiments, in some versions, (i) the spirulina comprises between 0.5X and 2X, (ii) the vitamin C comprises between 0.5Y and 2Y, and (iii) the spirulina comprises between 0.5Z and 2Z.

One variation is a combination of 452 mgs of spirulina, 298 mgs of vitamin C and 298 IU of vitamin D. In one version, the spirulina is Arthrospira platensis.

In some variations, the spirulina used is selected from locations in which the purity of the spirulina can be reliably expected to be high. Nonetheless, the formulation, kit and method disclosed herein, in some embodiments, has been checked by governmental authorities to ensure that it meets the quality, purity and other standards needed for medical use in regard to all three of the ingredients, for example in regard to minimizing the presence of heavy metals (i.e. lead, arsenic, cadmium, mercury) and certain microbes (E. coli, salmonella, staphylococcus, coliforms, yeast, molds, toxins etc.)

Typically, in the formulation there is less vitamin C (as measured in milligrams) and vitamin D (as measured in international units) than there is spirulina (as measured in milligrams). For example, the ratio of an amount of milligrams of vitamin C in the formulation to an amount of milligrams of spirulina in the formulation may be between 1:2 and 5:6. The ratio of the amount of IU of vitamin D in the formulation to an amount of milligrams of spirulina in the formulation may be between 1:2 and 5:6.

Another embodiment is a formulation comprising a combination of spirulina, vitamin C and vitamin D, wherein a ratio of an amount of milligrams of vitamin C in the formulation to an amount of milligrams of spirulina in the formulation is between 1:3 and 1:1, and a ratio of an amount of IU of vitamin Din the formulation to an amount of milligrams of spirulina in the formulation is between 1:3 and 1:1. In one variant of this embodiment, the ratio of the amount of milligrams of vitamin C in the formulation to the amount of milligrams of spirulina in the formulation is between 1:2 and 5:6 and wherein the ratio of the amount of IU of vitamin D in the formulation to the amount of milligrams of spirulina in the formulation is between 1:2 and 5:6. In another variant, the ratio of the amount of milligrams of vitamin C in the medicament to the amount of IU of vitamin D in the medicament to an amount of milligrams of spirulina in the medicament is between one-half and three-halves.

Another embodiment is a formulation comprising a combination of at least the following amounts of each of the following ingredients: 300 milligrams of spirulina; 200 milligrams of vitamin C; and 200 IU of vitamin D. For example the combination may comprise at least 400 milligrams of spirulina; at least 200 milligrams of vitamin C′ and at least 200 IU of vitamin D. In a different variant, the combination includes the following amounts of each of the following ingredients: 300 to 600 milligrams of spirulina; 200 to 400 milligrams of vitamin C; and 200 to 400 IU of vitamin D. In another variant, the combination includes the following amounts of each of the following ingredients: 400 to 500 milligrams of spirulina; 200 to 400 milligrams of vitamin C; and 200 to 400 IU of vitamin D.

In certain versions of the formulation, the amount of vitamin C may be 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 900 mg, 950 mg, or 1000 mg, or less than 450 mg, less than about 400 mg, less than 350 mg, less than 300 mg, less than 250 mg, or less than about 200 mg.

In certain versions of the formulation, the amount of vitamin D may be 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 900 mg, 950 mg, or 1000 mg, or less than about 500 IU, less than 450 IU, less than about 400 IU, less than 350 IU, less than 300 IU, less than 250 IU, less than about 200 IU.

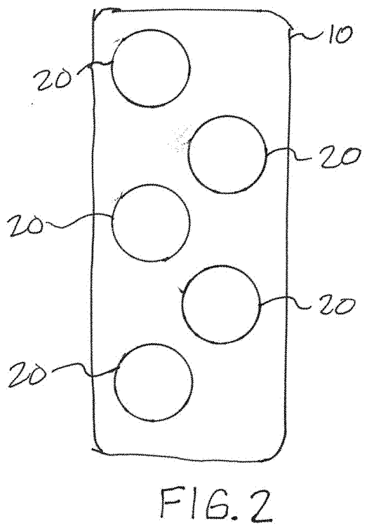

Another embodiment is a pharmaceutical pack or kit for preventing and/or treating a virus, such as a coronavirus such as a Covid-19 coronavirus (or for treating a pathogenic inflammation). As shown in FIG. 2, the kit 10 or pack 10 comprises or more vessels containing one or more compounds namely spirulina, vitamin C and vitamin D. Optionally associated with such container(s) can be a notice in the form prescribed by a governmental agency regulating the manufacture, use or sale of pharmaceutical or biological products, which reflects approval by the agency of manufacture, use or sale for human administration. As seen in FIG. 2, the kit or pack 10 may comprise: one or a combination of pills 20 (including but limited to tablets, caplets, capsules, pellets and/or lozenges) that in combination include at least: 452 milligrams of spirulina; 298 milligrams of vitamin C; and 298 IU of vitamin D. In one version of the kit or pack, the kit or pack includes one or a combination pills (including but not limited to tablets, caplets, capsules, pellets and/or lozenges) includes at least: 1356 milligrams of spirulina; 894 milligrams of vitamin C; and 894 IU of vitamin D.

The kit or pack may comprise one or a combination of tablets, caplets, capsules, pellets and/or lozenges, and these one or a combination of tablets, caplets, capsules, pellets and/or lozenges, may includes a total of 1300 to 3200 milligrams of spirulina, 894 to 2100 milligrams of vitamin C; and 894 to 2100 IU of vitamin D. In one non-limiting version, each of the tablets, caplets, capsules, pellets and/or lozenges in the kit or pack contains a combination of spirulina, vitamin C and vitamin D. In another non-limiting version, each of the tablets, caplets, capsules, pellets and/or lozenges in the kit or pack (or in a subvessel or subpackage of the kit or pack) contains spirulina and another of the tablets, caplets, capsules, pellets and/or lozenges in the kit or pack (or in a subvessel or subpackage of the kit or pack) contains vitamin C and another contains vitamin D.

In some embodiments, the kit or pack contains more than one combination of compounds. The kit or pack may optionally also contain an additional therapeutic agent.

Another embodiment is a method 100 of preventing and/or treating a Covid-19 coronavirus infection in a person. Method 100 may include a step 110 of administering to the person a daily dosage of a medicament that comprises spirulina, for example where the spirulina is Arthrospira platensis, vitamin C and vitamin D. In one version of method 100, (i) the spirulina comprises between 0.25X and 4X, wherein X is 452 milligrams, (ii) the vitamin C comprises between 0.25Y and 4Y, wherein Y is 298 milligrams, and (iii) the vitamin D comprises between 0.25Z and 4Z, wherein Z is 298 International Units (IU). In a second version using the same definitions of “X”, “Y” and “Z”, (i) the spirulina comprises between 0.5X and 2X, (ii) the vitamin C comprises between 0.5Y and 2Y, and (iii) the vitamin D comprises between 0.5Z and 2Z.

In versions of method 100, the daily dosage is administered to the person for 1 day, 2 days, 3 days, 4 days, 5 days, 6 days, 7 days, 8 days, 9 days, 10 days, 11 days, 12 days, 13 days or 14 days or more or for at least “X” days wherein “X” is an integer between 3 and 14. For example, in one non-limiting version the daily dosage is administered for (or at least for): 3-7 days or 3-8 days or 3-9 days or 3-10 days or 3-11 days or 3-12 days or 3-14 days.

The daily dosage of spirulina (in a formulation or in a kit or as part of method 100) may typically be 452 milligrams (times 1-6) because each medicament being administered (i.e. pill, including but not limited to a tablet, caplet, capsule, pellet, lozenge) may include in the formulation about 452 milligrams of spirulina but the patient or subject may have it administered once or more than once a day, for example he or she may take it once a day or twice a day or three times a day or four times a day or five times a day or six times a day (or more frequently). The medicament may be oral but may also be administered intravenously. Accordingly, the pill (including but not limited to a tablet, caplet, capsule, pellet, lozenge) or other medicament may include part or all of the daily dosage needed and may contain anywhere from between 300 to 400 milligrams to about 3200 mgs (or more) of spirulina.

The daily dosage of vitamin C (in a formulation or in a kit or as part of method 100) may typically be 298 milligrams (times 1-6) because each medicament being administered (i.e. pill, including but not limited to a tablet, caplet, capsule, lozenge, pellet) may comprise about 298 milligrams of vitamin C but the patient or subject may have it administered once or more than once a day, for example he or she may take it once a day or twice a day or three times a day or four times a day or five times a day or six times a day (or more frequently).

The dosage of vitamin C may be a low dosage, for example less than about 500 mg, less than 450 mg, less than about 400 mg, less than 350 mg, less than 300 mg, less than 250 mg, or less than about 200 mg. Accordingly, the medicament may be formulated to take into consideration part or all of the total daily dosage. Larger pills that have to be taken only once (for example for someone taking the maximum dosage) a day may be formulated.

The daily dosage of vitamin D (in a formulation or in a kit or as part of method 100) may typically be 298 IU (times 1-6) because each medicament being administered (i.e. pill, including but not limited to a tablet, lozenge, caplet, pellet, capsule) may comprise about 298 IU of vitamin D but the patient or subject may have it administered once or more than once a day, for example he or she may take it once a day or twice a day or three times a day or four times a day or five times a day or six times a day (or more frequently).

The dosage of vitamin D may be less than about 500 IU, less than 450 IU, less than about 400 IU, less than 350 IU, less than 300 IU, less than 250 IU, less than about 200 IU.

In method 100, the ratio of an amount of milligrams of vitamin C in the medicament to an amount of milligrams of spirulina in the medicament may be between 1:2 and 5:6. The ratio of the amount of IU of vitamin D in the medicament to an amount of milligrams of spirulina in the medicament may be between 1:2 and 5:6.

Another embodiment is a method of treating a pathogenic inflammation in a person. The method may include a step of administering to the person a daily dosage of a medicament that comprises spirulina, for example where the spirulina is Arthrospira platensis, vitamin C and vitamin D. All of the versions of the administering including for example the dosages, frequencies, concentrations, ratios and the time periods of the administering, described with respect to method 100 apply as well to this method (of treating a pathogenic inflammation).

### EXAMPLES

The following examples are not meant to limit the scope in any way. The following examples are put forth so as to provide those of ordinary skill in the art with a complete disclosure and description of how to make and use the described invention, and are not intended to limit the scope of the invention, nor are they intended to represent that the experiments below are all or the only experiments performed.

### Example 1—Clinical Study

A Helsinki committee approved questionnaire based clinical study was conducted in Israel on 94 non-hospitalized adult patients (a wide variety of ages from 18 through late sixties) diagnosed with Covid-19 and suffering from COVID-19 symptoms to evaluate the efficacy of a treatment regimen consisting of Vitamin, C, Spirulina, and Vitamin D (VSV). The regimen was taken for at least 3 days. The symptoms of these patients included difficulty breathing, fever, coughing, tiredness, muscle aches, weakness, loss of a sense of taste and smell. All but four of the adult patients used VSV in medicaments that had a specific proportion of 452 mg spirulina, 298 mg vitamin C and 298 IU vitamin D. The other four used it in a similar proportion whereby the amount of each ingredient may have varied from each of the 452, 298, 298 amounts by up to 10-20%.

In 40 of the patients, Covid-19 symptoms abated in 3 days or less. In 26 patients the Covid-19 symptoms abated in 3 days to 7 days (for a total of 66 patients whose symptoms abated in 7 days or less). In 12 other patients, the Covid-19 symptoms abated in 10-14 days (for a total of 78 patients whose symptoms abated in 14 days or less). In an additional 14 patients the patients reported that the symptoms abated but did not remember how long it took and/or whether they were sure if the VSV was the cause of the abatement. Two additional patients (out of the total of 94) did not cooperate in providing feedback.

None of the patients reported blood clots or neurological symptoms or other symptoms of “post-Covid” or reported hospitalization.

The results of the clinical study showed that oral administration of VSV stabilized the condition of symptomatic patients within 3 days in about 45% of reported cases and within 7 days in two-thirds of reported cases. In fact, significant improvement was noted in a high percentage of the patients during the period of the study. The median period of significant improvement for the 80 patients who reported recovery within a defined period of time was 3.25 days. This is a significantly shorter period of time than the publicly reported expected course of Covid-19.

Separately, a number of non-hospitalized patients suffering from asthma have taken a medicament comprising a combination of spirulina, vitamin C and vitamin D (in the proportion of 452 mg spirulina, 298 mg vitamin C and 298 IU vitamin D) for at least 3 days provided by their health care practitioners and have reported significant improvement with lower frequency of asthma attacks.

The term “pill” means tablet, capsule, caplet, pellet, lozenge.

“IU” is an abbreviation for International Units.

In some embodiments of the composition, in some embodiments of the kit or pack and in some embodiments of the methods, the spirulina is Arthrospira platensis.

The term “about” mean plus or minus 5%. For example, “about 50” means 47.5 to 52.5.

In some embodiments, the pills (including but not limited to the tablets, capsules, caplets, lozenges or pellets) used herein do not contain gluten, preservatives, sugar, sodium, yeast, artificial colors or artificial flavors.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method of using a medicament as a prophylactic against a phase of a Covid-19 coronavirus infection in a person, the phase comprising at least one of a neurological condition and a vascular condition, the method comprising:
administering to the person for at least three days a daily dosage of a medicament that comprises:
spirulina;
vitamin C; and
vitamin D,
wherein
(i) the spirulina comprises between 0.25X and 4X, wherein X is 452 milligrams,
(ii) the vitamin C comprises between 0.25Y and 4Y, wherein Y is 298 milligrams, and
(iii) the vitamin D comprises between 0.25Z and 4Z, wherein Z is 298 International Units (IU),
wherein a total daily amount of spirulina in the daily dosage is from one-half to 96% of the total daily amount of all active ingredients by weight.

2. The method of claim 1, wherein a total daily amount of spirulina in the daily dosage is from about 60% to 96% of the total daily amount of all active ingredients by weight.

3. The method of claim 1, wherein a total daily amount of spirulina in the daily dosage is from about 75% to 96% of the total daily amount of all active ingredients by weight.

4. A method of using a particular medicament as a prophylactic against both a respiratory phase of a Covid-19 coronavirus infection of a person during which the coronavirus is replicating and at least one of a post-viral neurological and post-viral vascular phase of the Covid-19 coronavirus infection in the person, comprising:
administering to the person, during the respiratory phase, for at least three days, a daily dosage of the particular medicament that comprises:
spirulina;
vitamin C; and
vitamin D,
wherein
(i) the spirulina comprises between 0.25X and 4X, wherein X is 452 milligrams,
(ii) the vitamin C comprises between 0.25Y and 4Y, wherein Y is 298 milligrams, and
(iii) the vitamin D comprises between 0.25Z and 4Z, wherein Z is 298 International Units (IU),
administering the particular medicament to the person during the post-viral neurological phase during which the person experiences neurological symptoms and/or during the post-viral vascular phase during which the subject experiences vascular symptoms, the daily dosage of the particular medicament for at least three other days.

5. The method of claim 4, wherein a ratio of an amount of milligrams of vitamin C in the medicament to an amount of milligrams of spirulina in the medicament is between 1:2 and 5:6.

6. The method of claim 4, wherein a ratio of the amount of IU of vitamin D in the medicament to an amount of milligrams of spirulina in the medicament is between 1:2 and 5:6.

7. The method of claim 4, wherein a total daily amount of spirulina in the daily dosage is from one-half to 96% of the total daily amount of all active ingredients by weight.

8. The method of claim 4, wherein a total daily amount of spirulina in the daily dosage is from 75% to 96% of the total daily amount of all active ingredients, by weight.

9. The method of claim 4, wherein a total daily amount of spirulina in the daily dosage is from 5/7 to about three-quarters of the total daily amount of all active ingredients by weight.

10. A method of treating both a respiratory phase of a Covid-19 coronavirus infection of a person, during which the coronavirus is replicating, using a particular medicament and at least one of a post-viral neurological and post-viral vascular phase of the Covid-19 coronavirus infection in the person using the particular medicament, comprising:
administering to the person, during the respiratory phase, for at least three days, a daily dosage of the particular medicament that comprises:
spirulina;
vitamin C; and
vitamin D,
wherein
(i) the spirulina comprises between 0.25X and 4X, wherein X is 452 milligrams,
(ii) the vitamin C comprises between 0.25Y and 4Y, wherein Y is 298 milligrams, and
(iii) the vitamin D comprises between 0.25Z and 4Z, wherein Z is 298 International Units (IU),
administering the particular medicament to the person during the post-viral neurological phase during which the person experiences neurological symptoms and/or during the post-viral vascular phase during which the subject experiences vascular symptoms, the daily dosage of the particular medicament for at least three other days.

11. The method of claim 10, wherein a ratio of an amount of milligrams of vitamin C in the medicament to an amount of milligrams of spirulina in the medicament is between 1:2 and 5:6.

12. The method of claim 10, wherein a ratio of the amount of IU of vitamin D in the medicament to an amount of milligrams of spirulina in the medicament is between 1:2 and 5:6.

13. The method of claim 10, wherein a total daily amount of spirulina in the daily dosage is from one-half to 96% of the total daily amount of all active ingredients by weight.

14. The method of claim 10, wherein a total daily amount of spirulina in the daily dosage is from 75% to 96% of the total daily amount of all active ingredients, by weight.

15. The method of claim 10, wherein a total daily amount of spirulina in the daily dosage is from 5/7 to about three-quarters of the total daily amount of all active ingredients by weight.

16. A method of treating a phase of a Covid-19 coronavirus infection in a person in which the person exhibits at least one of neurological conditions and vascular conditions, in a person, the method comprising:
administering to the person for at least three days a daily dosage of a medicament that comprises:
spirulina;
vitamin C; and
vitamin D,
wherein
(i) the spirulina comprises between 0.25X and 4X, wherein X is 452 milligrams,
(ii) the vitamin C comprises between 0.25Y and 4Y, wherein Y is 298 milligrams, and
(iii) the vitamin D comprises between 0.25Z and 4Z, wherein Z is 298 International Units (IU),
wherein a total daily amount of spirulina in the daily dosage is from one-half to 96% of the total daily amount of all active ingredients by weight.

17. The method of claim 16, wherein a total daily amount of spirulina in the daily dosage is from about 60% to 96% of the total daily amount of all active ingredients by weight.

18. The method of claim 16, wherein a total daily amount of spirulina in the daily dosage is from about 75% to 96% of the total daily amount of all active ingredients by weight.

19. A formulation comprising a combination of spirulina, vitamin C and vitamin D, wherein the spirulina comprises in milligrams X or a multiple of X, the vitamin C comprises in milligrams Y or a multiple of Y and the vitamin D comprises in International Units Z or a multiple of Z, wherein the multiple of X, the multiple of Y and the multiple of Z are identical multiples, wherein X is 300-600, Y is 200-400 and Z is 200-400, wherein the spirulina is more than 50% and up to 96% by weight of the formulation.

20. The formulation of claim 19, wherein the spirulina is more than about 60% and up to 96% by weight of the formulation.

21. The formulation of claim 19, wherein the spirulina is from about three-quarters to 96% by weight of the formulation.

22. The formulation of claim 19, wherein the spirulina is from about 70% to 96% by weight of the formulation.

23. The formulation of claim 19, wherein the spirulina is about 5/7 to about three-quarters by weight of the formulation.

24. The formulation of claim 19, wherein the ratio of the amount of milligrams of vitamin C in the formulation to the amount of milligrams of spirulina in the formulation is between 1:2 and 5:6.
